(19) United States
(12) Patent Application Publication
(10) Pub. No.: US 2009/0105366 A1
(43) Pub. Date: Apr. 23, 2009

(54) PASTE-LIKE POLYMETHYLMETHACRYLATE BONE CEMENT

(75) Inventors: SEBASTIAN VOGT, Erfurt (DE); HUBERT BUCHNER, Reinheim (DE)

Correspondence Address: NORRIS, MCLAUGHLIN & MARCUS, PA, 875 THIRD AVENUE, 18TH FLOOR, NEW YORK, NY, 10022, US

(73) Assignee: HERAEUS MEDICAL GMBH, Wehrheim (DE)

(21) Appl. No.: 12/254,516

(22) Filed: Oct. 20, 2008

(30) Foreign Application Priority Data
Oct. 22, 2007 (DE) 10 2007 050 762.5

(51) Int. Cl. A61L 24/00

(52) U.S. Cl. 523/116

(57) ABSTRACT

What is described is a paste-like PMMA bone cement that is characterised in that a paste-like component A, which is made up of one or more methacrylate monomers that can be distilled and polymerised by radical polymerisation, at least one polymer that is soluble in the methacrylate monomer/methacrylate monomers, at least one particulate polymer that is insoluble in the methacrylate monomer/methacrylate monomers and has a particle size of less than 500 μm, and at least one radical initiator, and a paste-like component B, which is made up of one or more methacrylate monomers that can be distilled and polymerised by radical polymerisation, at least one polymer that is soluble in the methacrylate monomer/methacrylate monomers, at least one particulate polymer that is insoluble in the methacrylate monomer/methacrylate monomers and has a particle size of less than 500 μm, and at least one accelerator, is present and in that a tack-free paste that cures by itself is generated upon mixing the paste-like components A and B.

The subject matter of the invention is a paste-like polymethylmethacrylate bone cement (PMMA bone cement).

PMMA bone cements have been known for decades and can be traced back to the ground-breaking work of Sir Charnley (Charnley, J.: Anchorage of the femoral head prosthesis of the shaft of the femur. J. Bone Joint Surg. 42 (1960) 28-30.). The principles of the basic structure of PMMA bone cements has remained unchanged since. PMMA bone cements consist of a liquid monomer component and a powder component. The monomer component generally contains the monomer, methylmethacrylate, and an activator (N,N-dimethyl-p-toluidine) dissolved therein. The powder component consists of one or more polymers that are made on the basis of methylmethacrylate and co-monomers, such as styrene, methylacrylate or similar monomers by polymerisation, preferably suspension polymerisation, a radio-opaquer and the initiator, dibenzoylperoxide. Mixing the powder component with the monomer component, swelling of the polymers of the powder component in the methylmethacrylate leads to the formation of a dough that can be deformed plastically. Simultaneously, the activator, N,N-dimethyl-p-toluidine, reacts with the dibenzoylperoxide which decomposes while forming radicals. The radicals thus formed initiate the radical polymerisation of the methylmethacrylate. Upon advancing polymerisation of the methylmethacrylate, the viscosity of the cement dough increases until the cement dough solidifies and is thus cured.

The fundamental mechanical requirements for PMMA bone cements, such as 4-point flexural strength, flexural modulus, and compressive strength are described in ISO 5833. To the user of the PMMA bone cements, the feature of the bone cement to be tack-free has essential importance. The term, tack-free, is defined in ISO5833. In conventional PMMA bone cements, being tack-free indicates that the cement has reached the processing phase through the swelling of the polymers contained in the cement powder in the monomer after the components are mixed. A PMMA bone cement must be tack-free as a matter of principle, to allow the user to shape and apply the cement. The PMMA bone cement must not adhere to the gloves and to application aids, such as mixing systems, crucibles or spatulas.

It is advantageous for PMMA bone cements to be dyed to allow the PMMA bone cement to be easily distinguished visually from bone tissue. For this reason, the PMMA bone cements of Heraeus Kulzer GmbH/Heraeus Medical GmbH have been dyed with copper-containing chlorophyll (chlorophyllin), which is known as food dye E141, for decades. DE102005033210 proposes a coloured PMMA bone cement that is characterised in that A a dye/dye mixture that is not or poorly soluble in methacrylic acid methylester and a synthetically produced, protein-free, hydrophobic, low-molecular or oligomeric solubiliser for the dye or dye mixture are dissolved in the monomer liquid, B in that the monomer liquid is clear to the view at room temperature, and C in that a dye/dye mixture that is not or poorly soluble in methacrylic acid methylester and a synthetically produced, protein-free, hydrophobic, low-molecular or oligomeric solubiliser are dissolved homogeneously in the polymethacrylate or polymethylmethacrylate. This means that a synthetic solubiliser allows dyes/dye mixtures that are inherently insoluble in methylmethacrylate to be used for homogeneous dyeing of the monomer component and of the powder component of PMMA bone cements. In contrast, DE102005032110 discloses a different variant for dyeing PMMA bone cements. It describes a dyed PMMA bone cement that is characterised in that at least the surface of the polymer particles of the powder component is partially or completely coated by a mixture of one or more dyes and a hydrophobic low-molecular or oligomeric organic bonding agent, where the quantity of bonding agent present is such that the polymer particles are not swollen as recognizable visually.

The essential disadvantage of the previous PMMA bone cements for the medical user is that the user needs to mix the liquid monomer component and the powder component in a mixing system or in crucibles right before application of the cement. Mixing errors with an adverse influence on the quality of the cement can easily occur in the process. The components must be mixed speedily. In the process, it is important that all of the cement powder is mixed with the monomer component without forming clumps and that the introduction of air bubbles during the mixing process is prevented. Unlike mixing by hand, the use of vacuum mixing systems largely prevents the formation of air bubbles in the cement dough, but an additional vacuum pump is required for these systems. Examples of mixing systems are disclosed in the specifications, U.S. Pat. No. 4,015,945, EP 0 674 888, and JP 2003181270. Vacuum mixing systems and vacuum pumps are relatively costly. After mixing the monomer component with the powder component, there is a need to wait for some, shorter or longer, time depending on the type of cement until the cement dough is tack-free and can be applied. Because of the many possibilities of errors that can occur during the mixing of conventional PMMA bone cements, appropriately trained personnel is needed. The training is associated with more than minor costs. Moreover, mixing the liquid monomer component with the powder component is associated with the user being exposed to stress in the form of monomer vapours and from the release of powder-like cement particles.

A significant disadvantage of the conventional PMMA bone cements for the cement producer is that both the powder component and the monomer component need to be produced such as to be packaged in a double-sterile manner. This means that at least four sterile packaging means are required for one package of bone cement.

It is therefore the object of the invention to develop a PMMA bone cement that alleviates or eliminates the above-mentioned disadvantages of the known PMMA bone cements. The PMMA bone cement to be developed shall, in particular, be provided for the user in a form such that the mixing of cement components, which is associated with many possibilities of errors, is avoided. The bone cement shall be as easy as possible to apply. The cement shall be provided such that a waiting phase until it is tack-free is not required. The viscosity and cohesion of the cement dough must be such that it withstands the bleeding pressure until it is cured. Moreover, the stress for the user from monomer vapours shall be avoided as much as possible.

The invention is based on the fundamental idea to dissolve, in a methacrylate monomer, a polymer that is soluble therein and suspend therein a particulate polymer that is insoluble in the methacrylate monomer. This enables the production of a dough-like mass that shows a high degree of internal cohesion due to the dissolved polymer, and has a sufficiently high viscosity due to the particulate insoluble polymer such that the dough can withstand the bleeding pressure for a short period of time. The dough can be cured by radical polymerisation of the methacrylate monomer. In this context, it is surprising that a weight ratio of methacrylate to soluble polymer to insoluble polymer was found at which the dough is tack-free. It is also essential that the dough is insoluble in water at this weight ratio of the components.

The object of the invention is therefore met by a paste-like PMMA bone cement that is characterised in that a paste-like component A, which is made up of one or more methacrylate monomers that can be distilled and polymerised by radical polymerisation, at least one polymer that is soluble in the methacrylate monomer/methacrylate monomers, at least one particulate polymer that is insoluble in the methacrylate monomer/methacrylate monomers and has a particle size of less than 500 μm, and at least one radical initiator, and a paste-like component B, which is made up of one or more methacrylate monomers that can be distilled and polymerised by radical polymerisation, at least one polymer that is soluble in the methacrylate monomer/methacrylate monomers, at least one particulate polymer that is insoluble in the methacrylate monomer/methacrylate monomers and has a particle size of less than 500 μm, and at least one accelerator, is present and in that a tack-free paste that cures by itself is generated upon mixing the paste-like components A and B.

It must be possible to distill the methacrylate monomers to allow cytotoxic contaminants that come up during monomer synthesis or remain in the monomer as non-reacted educts to definitely be removed from the monomer. In particular, residual methacrylic acid and methacrylic acid chloride have a cytotoxic effect.

The paste-like components A and B can be dyed by biocompatible dyes, whereby dyeing just one component is particularly advantageous in order to allow the mixing of the two components to be observable by eye.

The mixing of paste-like components A and B can be effected by simple kneading or mixing. It is feasible to store paste-like components A and B separately in twin-cartridges and mix them right before application by placing a static mixer on top of the twin-cartridge.

The particular advantage of the paste-like PMMA bone cement according to the invention is that no complicated mixing procedure needs to be performed and that no waiting phase until cement application is required after mixing. The cement can be applied right away after mixing.

It is useful for paste-like components A and B to have the same surface tension. This property is essential to allow homogenous mixing of paste-like components A and B to occur and to prevent segregation processes. The surface tension can be attained through the use of identical or similar monomers and through the use of identical or similar polymers in components A and B.

Difunctional methacrylates are preferred as methacrylate monomers, in particular ethylene glycol dimethacrylate, butan-1,3-diol-dimethacrylate, butan-1,4-diol-dimethacrylate, and hexan-1,6-diol-dimethacrylate. Moreover, it is also feasible to use other di- and trifunctional methacrylate monomers. It is also feasible to use monofunctional methacrylates that can be distilled. The scope of the invention also includes integrating additional monomers with bonding groups into the PMMA bone cement, such as, for example, methacrylic acid-2-hydroxyethylester. This can be used to exert a targeted influence on the bonding of the PMMA bone cement to the articular endoprostheses.

Poly-methylmethacrylate-copolymers are preferred as polymers that are soluble in the methacrylate monomer/methacrylate monomers, in particular poly-methylmethacrylate-co-methylacrylate and poly-methylmethacrylate-co-styrene. In addition, it is also feasible to use other copolymers that are made up of other alkylmethacrylates aside from methylmethacrylate.

It is desirable for the weight ratio of methylmethacrylate monomer/methylmethacrylate monomers to soluble polymer to polymer that is insoluble in the methylmethacrylate monomer/methylmethacrylate monomers to be such that the pasty components, A and B, are tack-free.

A weight ratio of 25-50 parts by weight methacrylate monomer/methacrylate monomers to 2-35 parts by weight soluble polymers and to 40-70 parts by weight polymer that is insoluble in the methylmethacrylate monomer/methylmethacrylate monomers is preferred.

Barbituric acid derivatives are preferred as radical initiator, in particular cyclohexylbarbituric acid. The term, barbituric acid derivatives, is meant to include alkyl, cycloalkyl, and aryl derivatives of barbituric acid, whereby the substituents are arranged in position 1 and 5 or only in position 5 of barbituric acid. This term also includes alkaline earth and alkaline salts of these barbituric acid derivatives. Moreover, peroxides, such as, e.g., dibenzoylperoxide, are also suitable as radical initiators.

Organic copper(II) salts are preferred as accelerators, in particular copper(II)-2-ethyl-hexanoate, copper(II) methacrylate, copper(II) hydroxide, and basic copper(II) carbonate. However, aside from these, it is also feasible to use N,N-dimethylaniline, N,N-dimethyl-p-toluidine, and N,N-bis(2-hydroxyethyl)-p-toluidine as accelerator—in case peroxides are used as initiator.

The radical initiator and the accelerator are usefully present in suitable concentrations for the cured bone cement not to be a source of cytotoxic effects on osteoblasts and osteoblast-like cells.

The paste-like PMMA bone cement can contain radio-opaquers, in particular zirconium dioxide, barium sulfate, tantalum, and biocompatible calcium salts.

The paste-like PMMA bone cement according to the invention can also contain pharmaceutical agents, whereby antibiotics, hormones, growth factors, and antiphlogistics are particularly preferred. Antibiotics to be considered are mainly aminoglycoside antibiotics, glycopeptide antibiotics, fluoroquinolone antibiotics, lincosamide antibiotics, and oxazolidinone antibiotics. Preferred in this context are gentamicin, tobramycin, amikacin, teicoplanin, vancomycin, ramoplanin, dalbavancin, moxifloxacin, ciprofloxacin, lincosamine, clindamycin, and linezolide. The antibiotics can be present in the PMMA bone cement according to the invention in particulate or in dissolved form.

Preferably, the paste-like PMMA bone cement contains one or more biocompatible elastomers that are particulate or soluble in the methacrylate monomer/methacrylate monomers, polybutadiene is particularly preferred.

The paste-like bone cement according to the invention is used, e.g., as self-curing plastic material for the fixation of total endoprostheses and revision endoprostheses and is particularly well-suited for the cementing of hip, knee, and shoulder total endoprostheses.

The paste-like bone cement according to the invention can also be used as self-curing filling material for vertebroplasty, kyphoplasty, and for femoral neck augmentation.

The paste-like PMMA bone cement according to the invention can also be used as self-curing plastic material for the production of local agent release systems. Accordingly, it is possible, e.g., to use a PMMA bone cement according to the invention that contains an antibiotic to form sphere-shaped or bean-shaped implants that can be used as local agent release systems.

The invention is illustrated by the examples presented in the following without limiting the scope of the invention. Like in the other parts of the description, specification of parts and percentages refers to the weight unless specified otherwise.

### EXAMPLE 1

### Synthesis of the calcium salt of 1-cyclohexyl-5-ethyl-barbituric acid (CaCHEBA)

A total of 10.000 g (42 mmol) 1-cyclohexyl-5-ethyl-barbituric acid and 1.621 g (21 mmol) calcium hydroxide were suspended in 50 ml methanol under stirring. Subsequently, stirring was continued for one hour at room temperature. Then, the methanol was removed using a vacuum rotary evaporator and the remaining residue was dried in a vacuum without any further cleaning operations until the mass was constant, whereby a colourless solid was obtained.

Yield: 11.000 g (97.8%)

FT-IR ν (cm⁻¹): 3211; 3134; 3083; 2940; 2857; 1748; 1711; 1664; 1427; 1364; 1319; 1260; 1207; 1136; 1088; 1075; 1043; 998; 896; 858; 805; 768; 754; 736; 717; 666.

### EXAMPLE 2

Production of a Mixture of Zirconium Dioxide and Copper Carbonate

A total of 20.000 g zirconium dioxide powder were mixed with 40 mg basic copper(II) carbonate (CuCO₃xCu(OH)₂) by intensive grinding.

### EXAMPLE 3

Production of a Mixture of Zirconium Dioxide and Copper Carbonate

A total of 10.000 g zirconium dioxide powder were mixed with 20 mg copper(II) hydroxide (stabilised Cu(OH)₂) by intensive grinding.

### EXAMPLE 4

Production of a Polymer Solution 1

A total of 15.0 g poly-methylmethacrylate-co-methylacrylate (molecular mass approx. 600,000; approx. 50% methylacrylate) were dissolved in 85.0 g hexan-1,6-diol-dimethacrylate at room temperature under intensive stirring. A viscous, clear solution was produced in the process.

### EXAMPLE 5

Production of a Polymer Solution 2

A total of 10.0 g poly-methylmethacrylate-co-methylacrylate (molecular mass approx. 600,000; approx. 50% methylacrylate) were dissolved in 80.0 g Hexan-1,6-diol-dimethacrylate at room temperature under intensive stirring. A viscous, clear solution was produced in the process.

A particulate poly-methylmethacrylate-co-methylacrylate (molecular mass approx. 800,000; approx. 5-8% methylacrylate, grain size <63 μm), hereinafter called polymer 1, was used for the pastes described in the following in examples 7-13. Moreover, a particulate, cross-linked poly-methylmethacrylate (Degacryl 6690) is used and hereinafter called polymer 2. Pastes A and B of examples 6-13 were produced by simple kneading of the components. Pastes A and B of examples 6-13 were tack-free and could be mixed without difficulty to form tack-free pastes 1-8, which subsequently cured by themselves.

### EXAMPLE 6

Paste 1

Paste A and paste B were tack-free, brush-applicable, visually homogeneous pastes that could be mixed with each other without difficulty.

Composition

| Paste components | Paste A | Paste B |
|---|---|---|
| Polymer 1 | 4.998 g | 5.250 g |
| Polymer solution 1 | 3.500 g | 3.500 g |
| Mixture of zirconium dioxide | 1.002 g | — |
| and copper carbonate | | |
| Zirconium dioxide | — | 1.000 g |
| CaCHEBA | 0.500 g | |
| 2-Ethyl-hexanoic acid | — | 0.200 g |
| ALIQUAT 336 | — | 0.050 g |

The paste generated after mixing of components A and B was easy to shape and brush-applicable without difficulty. The curing started 2 minutes and 50 seconds after the mixing.

### EXAMPLE 7

Paste 2

Composition

| Paste components | Paste A | Paste B |
|---|---|---|
| Polymer 1 | 4.998 g | 5.250 g |
| Polymer solution 1 | 3.500 g | 3.500 g |
| Mixture of zirconium dioxide | 0.501 g | — |
| and copper carbonate | | |
| Zirconium dioxide | 0.501 g | 1.000 g |
| CaCHEBA | 0.500 g | |
| 2-Ethyl-hexanoic acid | — | 0.200 g |
| ALIQUAT 336 | — | 0.050 g |

The curing started 4 minutes and 10 seconds after the mixing of components A and B.

### EXAMPLE 8

Paste 3

Composition

| Paste components | Paste A | Paste B |
|---|---|---|
| Polymer 1 | 4.998 g | 5.250 g |
| Polymer solution 1 | 3.500 g | 3.500 g |
| Mixture of zirconium dioxide | 0.250 g | — |
| and copper carbonate | | |
| Zirconium dioxide | 0.752 g | 1.000 g |
| CaCHEBA | 0.500 g | |
| 2-Ethyl-hexanoic acid | — | 0.200 g |
| ALIQUAT 336 | — | 0.050 g |

The curing started 6 minutes and 15 seconds after the mixing.

### EXAMPLE 9

Paste 4

Composition

| Paste components | Paste A | Paste B |
|---|---|---|
| Polymer 1 | 4.998 g | 5.250 g |
| Polymer solution 1 | 3.500 g | 3.500 g |
| Mixture of zirconium dioxide | 1.002 g | — |
| and copper carbonate | | |
| Zirconium dioxide | — | 1.000 g |
| CaCHEBA | 0.500 g | |
| Octanoic acid | — | 0.200 g |
| ALIQUAT 336 | — | 0.050 g |

After mixing of components A and B, the paste again was easy to shape and apply with a brush without difficulty. The curing started 3 minutes and 5 seconds after the mixing.

### EXAMPLE 10

Paste 5

Composition

| Paste components | Paste A | Paste B |
|---|---|---|
| Polymer 1 | 4.998 g | 5.250 g |
| Polymer solution 1 | 3.500 g | 3.500 g |
| Mixture of zirconium dioxide | 1.002 g | — |
| and copper carbonate | | |
| Zirconium dioxide | — | 1.000 g |
| CaCHEBA | 0.500 g | |
| Heptanoic acid | — | 0.200 g |
| ALIQUAT 336 | — | 0.050 g |

After mixing of components A and B, the paste again was easy to shape and apply with a brush without difficulty. The curing started 3 minutes and 5 seconds after the mixing.

### EXAMPLE 11

Paste 6

Composition

| Paste components | Paste A | Paste B |
|---|---|---|
| Polymer 1 | 4.998 g | 5.250 g |
| Polymer solution 1 | 3.500 g | 3.500 g |
| Mixture of zirconium dioxide | 0.501 g | — |
| and copper hydroxide | | |
| Zirconium dioxide | 0.501 g | 1.000 g |
| CaCHEBA | 0.500 g | |
| Heptanoic acid | — | 0.200 g |
| ALIQUAT 336 | — | 0.050 g |

After mixing of components A and B, the paste again was easy to shape and apply with a brush without difficulty. The curing started 3 minutes and 20 seconds after the mixing.

### EXAMPLE 12

Paste 7

Composition

| Paste components | Paste A | Paste B |
|---|---|---|
| Polymer 1 | 4.998 g | 5.250 g |
| Polymer solution 2 | 3.500 g | 3.500 g |
| Mixture of zirconium dioxide | 0.501 g | — |
| and copper hydroxide | | |
| Zirconium dioxide | 0.501 g | 1.000 g |
| CaCHEBA | 0.500 g | |
| 2-Ethyl-hexanoic acid | — | 0.200 g |
| ALIQUAT 336 | — | 0.050 g |

After mixing of components A and B, the paste again was easy to shape and apply with a brush without difficulty. The curing started 4 minutes and 25 seconds after the mixing.

### EXAMPLE 13

Paste 8

Composition

| Paste components | Paste A | Paste B |
|---|---|---|
| Polymer 2 | 4.998 g | 5.250 g |
| Polymer solution 1 | 3.500 g | 3.500 g |
| Mixture of zirconium dioxide | 0.501 g | — |
| and copper carbonate | | |
| Zirconium dioxide | 0.501 g | 1.000 g |
| CaCHEBA | 0.500 g | |
| 2-Ethyl-hexanoic acid | — | 0.200 g |
| ALIQUAT 336 | — | 0.050 g |

The curing started 4 minutes and 5 seconds after the mixing of components A and B.

1. Paste-like PMMA bone cement comprising
(a) first a paste-like component, comprising
(i) a first methacrylate monomer that can be distilled and polymerised by radical polymerisation;
(ii) polymer that is soluble in said first methacrylate monomer;
(iii) at least one particulate polymer that is insoluble in said first methacrylate monomer and has a particle size of no more than 500 μm; and
(iv) at least one radical initiator;
and a (b) second paste-like components, comprising
(i) a second methacrylate monomer that can be distilled and polymerised by radical polymerisation;
(ii) at least one polymer that is soluble in said second methacrylate monomer;
(iii) at least one particulate polymer that is insoluble in said second methacrylate monomer and has a particle size of less than 500 μm;
(iv) and at least one accelerator.

2. Paste-like PMMA bone cement according to claim 1, wherein the first and second paste-like components have the same surface tension.

3. Paste-like PMMA bone cement according to claim 1, wherein the first and second methacrylate monomers are mono- or difunctional methacrylates.

4. Paste-like PMMA bone cement according to claim 1, wherein the first and second methacrylate monomers are selected from the group consisting of ethylene glycol dimethacrylate, butan-1,3-diol-dimethacrylate, butan-1,4-diol-dimethacrylate, and hexan-1,6-diol-dimethacrylate.

5. Paste-like PMMA bone cement according to claim 1, wherein the soluble polymers are poly-methylmethacrylate-copolymers.

6. Paste-like PMMA bone cement according to claim 5, wherein the first and second polymers are poly-methylmethacrylate-co-methylacrylate or poly-methylmethacrylate-co-styrene.

7. Paste-like PMMA bone cement according to claim 1, wherein the first and second insoluble polymers are cross-linked poly-methylmethacrylate or cross-linked poly-methylmethacrylate-co-methylacrylate.

8. Paste-like PMMA bone cement according to claim 1, wherein the weight ratios of the first and second soluble polymer and the first and second insoluble polymer are selected such that the paste-like components are tack-free.

9. Paste-like PMMA bone cement according to claim 1, comprising 25-50 parts by weight of the methacrylate monomers collectively, the soluble polymers collectively, and 40-70 parts by weight the insoluble polymers collectively.

10. Paste-like PMMA bone cement according to claim 1, wherein barbituric acid derivates are used as radical initiator.

11. Paste-like PMMA bone cement according to claim 10, wherein 1-cyclohexyl-5-ethyl-barbituric acid or the calcium salt of 1-cyclohexyl-5-ethyl-barbituric acid is used as radical initiator.

12. Paste-like PMMA bone cement according to claim 1, wherein organic copper(II) salts are used as accelerator.

13. Paste-like PMMA bone cement according to claim 12, wherein copper(II) 2-ethyl-hexanoate, copper(II) methacrylate, basic copper(II) carbonate or copper(II) hydroxide is used as accelerator.

14. Paste-like PMMA bone cement according to claim 1 further comprising radio-opaquers selected from the group consisting of zirconium dioxide, barium sulfate, tantalum, and biocompatible calcium salts.

15. Paste-like PMMA bone cement according to claim 1 further comprising pharmaceutical agents selected from the groups consisting of antibiotics, hormones, growth factors, and antiphlogistics.

16. Paste-like PMMA bone cement according to claim 1 further comprising one or more biocompatible elastomers that are particulate or soluble in the methacrylate monomer/methacrylate monomers.

17. Paste-like PMMA bone cement according to claim 16, wherein the elastomer is polybutadiene.

18. (canceled)

19. (canceled)

20. (canceled)
